(19) United States
(12) Patent Application Publication
(10) Pub. No.: US 2021/0187149 A1
(43) Pub. Date: Jun. 24, 2021

(54) DISTRIBUTING LIGHT IN A REACTION CHAMBER

(71) Applicants: ACUVA TECHNOLOGIES INC., Burnaby (CA); THE UNIVERSITY OF BRITISH COLUMBIA, Vancouver (CA)

(72) Inventors: Babak ADELI KOUDEHI, Vancouver (CA); Fariborz TAGHIPOUR, Burnaby (CA); Manoj SINGH, Vancouver, (CA); Alireza JALALI, West Vancouver, (CA)

(21) Appl. No.: 16/650,148

(22) PCT Filed: Sep. 25, 2018
(86) PCT No.: PCT/CA2018/051212

§ 371 Date: Mar. 24, 2020

(30) Foreign Application Priority Data
Sep. 25, 2017 (CA) 2980178

(51) Int. Cl. A61L 9/20; C02F 1/32

(52) U.S. Cl. CPC A61L 9/20; C02F 2201/3222; C02F 1/325

(57) ABSTRACT

Distributing electromagnetic radiation in a reaction chamber may involve causing at least some electromagnetic radiation from at least one electromagnetic radiation emitter to be refracted by at least one lens into the reaction chamber as refracted electromagnetic radiation skewed laterally relative to a longitudinal direction of the reaction chamber and/or relative to the at least some electromagnetic radiation from the at least one electromagnetic radiation emitter.

### CROSS-REFERENCE TO RELATED APPLICATION

This application claims the benefit of, and priority to, Canadian patent application no. 2,980,178 filed Sep. 25, 2017, the entire contents of which are incorporated by reference herein.

### FIELD

This disclosure relates generally to distributing light in a reaction chamber.

### RELATED ART

Fluids, such as water or air for example, may be treated, for example to deactivate pathogens, by subjecting the fluid to ultraviolet (“UV”) light in a reaction chamber. Solid-state light sources such as light-emitting diodes (“LEDs”) may produce such UV light, but such light may not be adequately distributed throughout a reaction chamber.

As a result, a reaction chamber may have one or more dark regions exposed to little or no such light. For example, a fully collimated or converging-collimated radiation pattern may conserve power, but may leave dark regions that may lead to decrease in reactor performance, particularly when the reaction chamber consists of one channel only.

Similarly, when local fluid velocity is higher in some locations of a reaction chamber, for example due to introduction of fluid to the reaction chamber from a side of the reaction chamber, fluid at such a higher fluid velocity requires higher UV intensity to reach to similar level of disinfection when compared to fluid having a lower velocity.

Pathogens in fluid passing through such dark regions, or flowing with such high-velocity fluid, may not be deactivated, which may be hazardous to health.

### SUMMARY

According to one embodiment, there is provided a method of distributing electromagnetic radiation in a reaction chamber extending in a longitudinal direction at least between an inlet of the reaction chamber and an outlet of the reaction chamber, the method comprising causing at least some electromagnetic radiation from at least one electromagnetic radiation emitter to be refracted by at least one lens into the reaction chamber as refracted electromagnetic radiation skewed laterally relative to the longitudinal direction.

According to another embodiment, there is provided a method of distributing electromagnetic radiation in a reaction chamber, the method comprising causing at least some electromagnetic radiation from at least one electromagnetic radiation emitter to be refracted by at least one lens into the reaction chamber as refracted electromagnetic radiation skewed laterally relative to the at least some electromagnetic radiation from the at least one electromagnetic radiation emitter.

In some embodiments, the reaction chamber extends in a longitudinal direction at least between an inlet of the reaction chamber and an outlet of the reaction chamber.

In some embodiments, causing the at least some of the refracted electromagnetic radiation to be refracted into the reaction chamber comprises causing the refracted electromagnetic radiation in the reaction chamber to be skewed laterally relative to the longitudinal direction.

In some embodiments, the longitudinal direction is parallel to a central longitudinal axis of the reaction chamber.

In some embodiments, the inlet is configured to direct fluid into the reaction chamber in an inlet direction non-parallel to the longitudinal direction.

In some embodiments, the inlet direction is substantially perpendicular to the longitudinal direction.

In some embodiments, causing the at least some of the refracted electromagnetic radiation to be refracted into the reaction chamber comprises causing fluence rate of the refracted electromagnetic radiation in the reaction chamber and along the inlet direction from the inlet to be higher with increased distance from the inlet.

In some embodiments, causing the at least some of the refracted electromagnetic radiation to be refracted into the reaction chamber comprises causing a fluence rate of the refracted electromagnetic radiation in a first transverse side of the reaction chamber proximate the inlet to be less than a fluence rate of the refracted electromagnetic radiation in a second transverse side of the reaction chamber opposite the first transverse side of the reaction chamber and opposite the inlet.

In some embodiments, the inlet is configured to direct fluid into the reaction chamber in an inlet direction substantially parallel to the longitudinal direction.

In some embodiments, causing the refracted electromagnetic radiation in the reaction chamber to be skewed laterally relative to the longitudinal direction comprises causing the refracted electromagnetic radiation in the reaction chamber to be skewed laterally relative to the longitudinal direction and towards an extension in the reaction chamber of the inlet direction from the inlet.

In some embodiments, causing the at least some electromagnetic radiation from the at least one electromagnetic radiation emitter to be refracted by the at least one lens into the reaction chamber comprises causing the at least some electromagnetic radiation from the at least one electromagnetic radiation emitter to be: refracted into the reaction chamber by a plurality of lenses spaced apart around an inlet axis extending along the inlet direction; and skewed laterally relative to the longitudinal direction and towards the extension in the reaction chamber of the inlet direction from the inlet.

In some embodiments, the plurality of lenses surround the inlet axis.

In some embodiments, the electromagnetic radiation comprises ultraviolet (“UV”) radiation.

In some embodiments, the at least one electromagnetic radiation emitter comprises at least one UV light-emitting diode (“UV-LED”).

In some embodiments, the at least one electromagnetic radiation emitter comprises at least one light-emitting diode (“LED”).

In some embodiments, the at least some electromagnetic radiation from the at least one electromagnetic radiation emitter has a principal radiation direction.

In some embodiments, the at least some electromagnetic radiation from the at least one electromagnetic radiation emitter is substantially axially symmetric about the principal radiation direction.

In some embodiments, the refracted electromagnetic radiation is distributed axially asymmetrically relative to the principal radiation direction of the at least some electromagnetic radiation from the at least one electromagnetic radiation emitter. In some embodiments, a fluence rate of the refracted electromagnetic radiation on a first transverse side of the principal radiation direction is greater than a fluence rate of the refracted electromagnetic radiation on a second transverse side of the principal radiation direction opposite the first transverse side of the principal radiation direction.

In some embodiments, the at least one lens comprises at least one lens having an optical axis non-parallel to the principal radiation direction.

In some embodiments, the at least one lens comprises at least one lens having an optical axis parallel to and spaced apart from the principal radiation direction.

In some embodiments, the at least one lens comprises at least one axially asymmetric lens.

According to another embodiment, there is provided a reactor apparatus comprising: a body defining an inlet, an outlet, and a reaction chamber extending in a longitudinal direction at least between the inlet and the outlet; at least one electromagnetic radiation emitter; and at least one lens configured to refract at least some electromagnetic radiation from the at least one electromagnetic radiation emitter into the reaction chamber as refracted electromagnetic radiation skewed laterally relative to the longitudinal direction.

According to another embodiment, there is provided a reactor apparatus comprising: a body defining a reaction chamber; at least one electromagnetic radiation emitter; and at least one lens configured to refract at least some electromagnetic radiation from the at least one electromagnetic radiation emitter into the reaction chamber as refracted electromagnetic radiation skewed laterally relative to the at least some electromagnetic radiation from the at least one electromagnetic radiation emitter and into the reaction chamber.

In some embodiments: the body further defines an inlet of the reaction chamber and an outlet of the reaction chamber; and the reaction chamber extends in a longitudinal direction at least between the inlet and the outlet.

In some embodiments, the at least one lens is configured to cause the refracted electromagnetic radiation in the reaction chamber to be skewed laterally relative to the longitudinal direction.

In some embodiments, the longitudinal direction is parallel to a central longitudinal axis of the reaction chamber.

In some embodiments, the inlet is configured to direct fluid into the reaction chamber in an inlet direction non-parallel to the longitudinal direction.

In some embodiments, the inlet direction is substantially perpendicular to the longitudinal direction.

In some embodiments, the at least one lens is configured to cause fluence rate of the refracted electromagnetic radiation in the reaction chamber and along the inlet direction from the inlet to be higher with increased distance from the inlet.

In some embodiments, the at least one lens is configured to cause a fluence rate of the refracted electromagnetic radiation in a first transverse side of the reaction chamber proximate the inlet to be less than a fluence rate of the refracted electromagnetic radiation in a second transverse side of the reaction chamber opposite the first transverse side of the reaction chamber and opposite the inlet.

In some embodiments, the inlet is configured to direct fluid into the reaction chamber in an inlet direction substantially parallel to the longitudinal direction.

In some embodiments, the at least one lens is configured to cause the refracted electromagnetic radiation in the reaction chamber to be skewed laterally relative to the longitudinal direction and towards an extension in the reaction chamber of the inlet direction from the inlet.

In some embodiments, the at least one lens comprises a plurality of lenses spaced apart around an inlet axis extending along the inlet direction.

In some embodiments, the plurality of lenses surround the inlet axis.

In some embodiments, the at least one electromagnetic radiation emitter comprises at least one emitter of UV radiation.

In some embodiments, the at least one emitter of UV radiation comprises at least one UV-LED.

In some embodiments, the at least one electromagnetic radiation emitter comprises at least one LED.

In some embodiments, the at least one electromagnetic radiation emitter is configured to cause the at least some electromagnetic radiation from the at least one electromagnetic radiation emitter to have a principal radiation direction.

In some embodiments, the at least one electromagnetic radiation emitter is configured to cause the at least some electromagnetic radiation from the at least one electromagnetic radiation emitter to be substantially axially symmetric about the principal radiation direction.

In some embodiments, the at least one lens is configured to cause the refracted electromagnetic radiation to be distributed axially asymmetrically relative to the principal radiation direction of the at least some electromagnetic radiation from the at least one electromagnetic radiation emitter. In some embodiments, the at least one lens is configured to cause a fluence rate of the refracted electromagnetic radiation on a first transverse side of the principal radiation direction to be greater than a fluence rate of the refracted electromagnetic radiation on a second transverse side of the principal radiation direction opposite the first transverse side of the principal radiation direction. In some embodiments, the at least one lens has an optical axis non-parallel to the principal radiation direction.

In some embodiments, the at least one lens has an optical axis parallel to and spaced apart from the principal radiation direction.

In some embodiments, the at least one lens comprises an axially asymmetric lens. Other aspects and features will become apparent to those ordinarily skilled in the art upon review of the following description of illustrative embodiments in conjunction with the accompanying figures.

### BRIEF DESCRIPTION OF THE DRAWINGS

FIG. 1 is a perspective view of a reactor apparatus according to one embodiment.

FIG. 2 is a cross-sectional view of the reactor apparatus of FIG. 1, taken along the line 2-2 in FIG. 1.

FIG. 3 is a cross-sectional view of a reactor head of the reactor apparatus of FIG. 1.

FIG. 4 is a cross-sectional view of a reactor head according to another embodiment.

FIG. 5 is a cross-sectional view of a reactor head according to another embodiment.

FIG. 6 is a cross-sectional view of a reactor head according to another embodiment.

FIG. 7 is a cross-sectional view of a reactor head according to another embodiment.

FIG. 8 is a cross-sectional view of a reactor head according to another embodiment.

FIG. 9 is a cross-sectional view of a reactor head according to another embodiment.

FIG. 10 is a perspective view of a reactor head according to another embodiment.

FIG. 11 is a side view of the reactor head of FIG. 10.

FIG. 12 is a perspective view of a reactor head according to another embodiment.

FIG. 13 is a side view of the reactor head of FIG. 12.

FIG. 14 is a perspective view of a reactor head according to another embodiment.

FIG. 15 is a cross-sectional view of a reactor apparatus according to another embodiment.

FIG. 16 is a cross-sectional view of a reactor apparatus according to another embodiment.

### DETAILED DESCRIPTION

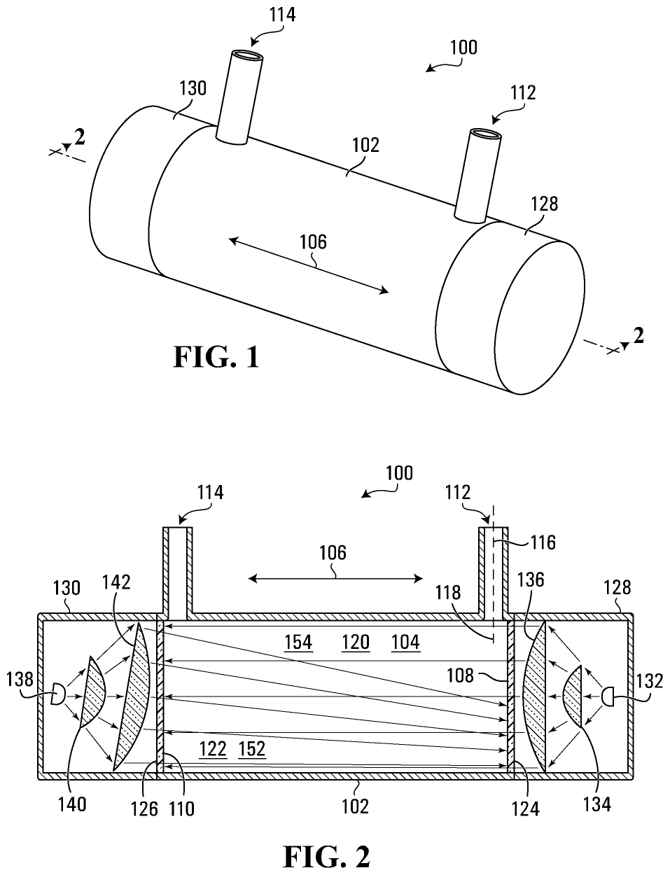

Referring to FIGS. 1 and 2, a reactor apparatus according to one embodiment is shown generally at 100 and includes a reactor body 102 that defines a reaction chamber 104 that extends in a longitudinal direction 106 between longitudinal ends 108 and 110 of the reaction chamber 104. The reactor body 102 also defines an inlet 112 of the reaction chamber 104 proximate the longitudinal end 108 and an outlet 114 of the reaction chamber 104 proximate the longitudinal end 110. The reaction chamber 104 therefore extends in the longitudinal direction 106 at least between the inlet 112 and the outlet 114.

The inlet 112 extends along an inlet axis 116 and is therefore configured to direct fluid into the reaction chamber 104 in an inlet direction 118 that may be an extension of the inlet axis 116 into the reaction chamber 104 and may be substantially perpendicular to the longitudinal direction 106. However, the inlet direction 118 may differ in other embodiments and may, for example, be in other directions non-parallel to the longitudinal direction 106. The reaction chamber 104 has a transverse side 120 proximate the inlet 112, and a transverse side 122 opposite the transverse side 120 and opposite the inlet 112. In the embodiment shown, because the inlet direction 118 is non-parallel to the longitudinal direction 106, fluid in the reaction chamber 104 may flow faster in regions of the reaction chamber 104 that are downstream from the inlet 112 than in other regions of the reaction chamber 104, and fluid in the reaction chamber 104 may flow faster in the transverse side 122 than in the transverse side 120.

The reactor apparatus 100 includes a translucent or transparent wall 124 at the longitudinal end 108, and a translucent or transparent wall 126 at the longitudinal end 110. The reactor apparatus 100 also includes a reactor head 128 proximate the longitudinal end 108 and positioned to direct electromagnetic radiation through the translucent or transparent wall 124 and into the reaction chamber 104 from the longitudinal end 108. The reactor apparatus 100 also includes a reactor head 130 proximate the longitudinal end 110 and positioned to direct electromagnetic radiation through the translucent or transparent wall 126 and into the reaction chamber 104 from the longitudinal end 110. Therefore, the translucent or transparent walls 124 and 126 may be translucent or transparent electromagnetic radiation from different reactor heads such as those described herein, for example.

The reactor head 128 includes a UV light-emitting diode (“UV-LED”) 132, a lens 134, and a lens 136. In the embodiment shown, the lens 134 is a half-ball lens and the lens 136 is a plano-convex lens, although alternative embodiments may differ. At least some UV radiation from the UV-LED 132 may be refracted by the lens 134, at least some UV radiation refracted by the lens 134 may be refracted by the lens 136, and at least some UV radiation refracted by the lens 136 may be directed through the translucent or transparent wall 124 and into the reaction chamber 104 from the longitudinal end 108. Therefore, the UV-LED 132, the lens 134, and the lens 136 may collectively function as a UV source (or, more generally, as an electromagnetic radiation source) for a reaction chamber such as reaction chambers described herein, for example. As shown in FIG. 2, such UV radiation refracted from the UV-LED 132 and into the reaction chamber 104 from the longitudinal end 108 may be substantially collimated or may be divergent, and a principal radiation direction of such UV radiation refracted from the UV-LED 132 and into the reaction chamber 104 may be substantially parallel to the longitudinal direction 106. However, alternative embodiments may differ.

In general, a principal radiation direction of electromagnetic radiation may be an intensity-weighted average direction of travel of the electromagnetic radiation or may be defined in other ways. In general, electromagnetic radiation may be axially symmetric or may be axially asymmetric about its principal radiation direction.

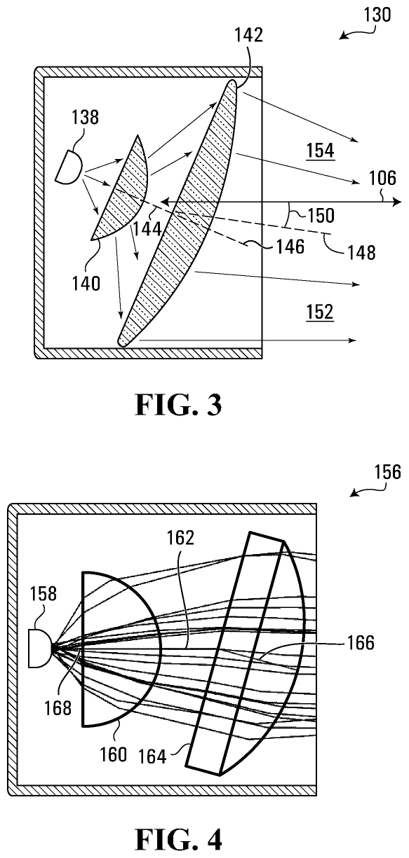

Referring to FIGS. 2 and 3, the reactor head 130 includes a UV-LED 138, a lens 140, and a lens 142. In the embodiment shown, the lens 140 is a half-ball lens and the lens 142 is a plano-convex lens, although alternative embodiments may differ. At least some UV radiation from the UV-LED 138 may be refracted by the lens 140, at least some UV radiation refracted by the lens 140 may be refracted by the lens 142, and at least some UV radiation refracted by the lens 142 may be directed through the translucent or transparent wall 126 and into the reaction chamber 104 from the longitudinal end 110. Therefore, the UV-LED 138, the lens 140, and the lens 142 may collectively function as a UV source (or, more generally, as an electromagnetic radiation source) for a reaction chamber such as reaction chambers described herein, for example.

As shown in FIG. 3, the lens 140 has an optical axis 144, and the lens 142 has an optical axis 146. Further, in the embodiment shown, the optical axes 144 and 146 are substantially collinear, and UV radiation from the UV-LED 138 may be substantially axially symmetric about the optical axis 144, although alternative embodiments may differ. However, the optical axes 144 and 146 are non-parallel and oblique to the longitudinal direction 106. In the embodiment shown, an oblique angle between the optical axes 144 and 146 and the longitudinal direction 106 may be between about 1 degree and about 45 degrees, although alternative embodiments may differ. As a result, as shown in FIGS. 2 and 3, UV radiation refracted from the UV-LED 138 and into the reaction chamber 104 from the longitudinal end 110 is skewed laterally relative to the longitudinal direction 106, and a principal radiation direction 148 of the UV radiation refracted by the lens 142 is an oblique angle 150 from the longitudinal direction 106.

As shown in FIG. 3, the UV radiation refracted from the UV-LED 138 and into the reaction chamber 104 from the longitudinal end 110 is skewed laterally relative to the UV radiation refracted from the UV-LED 138 in a transverse direction away from the inlet 112. As a result, along the inlet direction 118 from the inlet 112, a fluence rate (density of intensity) or local intensity of the UV radiation refracted from the UV-LED 138 and into the reaction chamber 104 from the longitudinal end 110 increases with increased distance from the inlet 112. Also, as a result, a fluence rate or local intensity of the UV radiation refracted from the UV-LED 138 and into the transverse side 120 of the reaction chamber 104 from the longitudinal end 110 is less than a fluence rate or local intensity of the UV radiation refracted from the UV-LED 138 and into the transverse side 122 of the reaction chamber 104 from the longitudinal end 110.

As indicated above, in the embodiment shown, fluid in the reaction chamber 104 may flow faster in regions of the reaction chamber 104 that are downstream from the inlet 112 than in other regions of the reaction chamber 104, and fluid in the reaction chamber 104 may flow faster in the transverse side 122 than in the transverse side 120. As shown in FIG. 2, because the UV radiation refracted from the UV-LED 138 and into the reaction chamber 104 from the longitudinal end 110 is skewed laterally in a transverse direction away from the inlet 112, UV radiation fluence rate or local intensity in the reaction chamber 104 may correlate with fluid flow velocity in the reaction chamber 104. In other words, in general, UV radiation fluence rate or local intensity in the reaction chamber 104 may be higher in regions where fluid flow velocity in the reaction chamber 104 may also be higher, and total UV exposure to fluid flowing through the reaction chamber 104 may be more consistent than in other reactor apparatuses without such skewed UV radiation.

Referring to FIG. 4, a reactor head according to another embodiment is shown generally at 156 and includes a UV-LED 158, a lens 160 having an optical axis 162, and a lens 164 having an optical axis 166. In the embodiment shown, the lens 160 is a half-ball lens and the lens 164 is a plano-convex lens, although alternative embodiments may differ. At least some UV radiation from the UV-LED 158 may be refracted by the lens 160, at least some UV radiation refracted by the lens 160 may be refracted by the lens 164, and at least some UV radiation refracted by the lens 164 may be directed into a reaction chamber, for example through the translucent or transparent wall 124 and into the reaction chamber 104 from the longitudinal end 108 or through the translucent or transparent wall 126 and into the reaction chamber 104 from the longitudinal end 110, or more generally into one or both longitudinal ends of a reaction chamber such as reaction chambers as described herein. Therefore, the UV-LED 158, the lens 160, and the lens 164 may collectively function as a UV source (or, more generally, as an electromagnetic radiation source) for a reaction chamber such as reaction chambers described herein, for example.

The UV radiation from the UV-LED 158 may be substantially axially symmetric about a principal radiation direction 168, and the optical axis 162 is substantially collinear with the principal radiation direction 168, although alternative embodiments may differ. However, the optical axis 166 is non-parallel and oblique to the principal radiation direction 168 and to the optical axis 162. In the embodiment shown, an oblique angle between the optical axis 166 and the principal radiation direction 168 (or between the optical axis 166 and a longitudinal direction of a reaction chamber, such as the longitudinal direction 106 of the reaction chamber 104, for example) may be between about 1 degree and about 45 degrees, although alternative embodiments may differ. As a result, UV radiation refracted by the lens 164 is not substantially axially symmetric about the principal radiation direction 168, but is rather skewed laterally relative to the principal radiation direction 168 and skewed laterally relative to the UV radiation refracted from the UV-LED 158. In other words, a fluence rate or local intensity of the UV radiation from the UV-LED 158 and refracted by the lenses 160 and 164 is greater on one transverse side of the principal radiation direction 168 (above the principal radiation direction 168 in the orientation of FIG. 4) than on an opposite transverse side of the principal radiation direction 168 (below the principal radiation direction 168 in the orientation of FIG. 4). Further, UV radiation refracted by the lens 164 may be refracted into a reaction chamber, and UV radiation refracted by the lens 164 and into a reaction chamber may be skewed laterally relative to a longitudinal direction of the reaction chamber, similarly to the reactor head 130 as described above with reference to FIG. 2, for example.

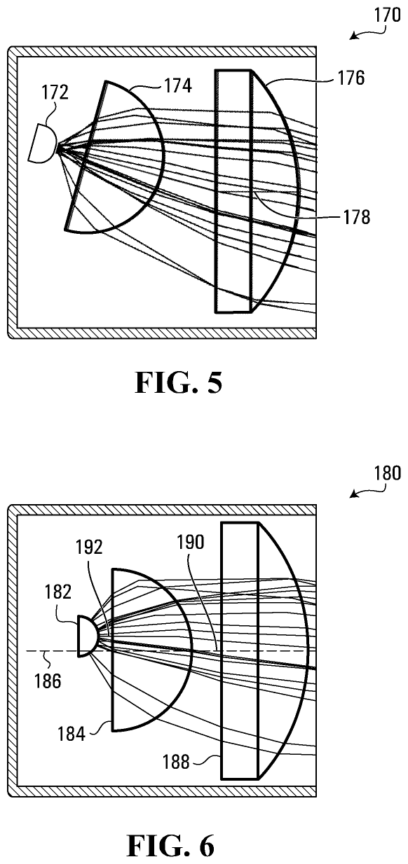

In the embodiment of FIG. 4, the UV-LED 158, the lens 160, and the lens 164 may be positioned in the reactor head 156 such that the principal radiation direction 168 and the optical axis 162 may be parallel to a longitudinal direction of a reaction chamber (such as the longitudinal direction 106 of the reaction chamber 104, for example), but alternative embodiments may differ. For example, referring to FIG. 5, a reactor head according to another embodiment is shown generally at 170 and includes a UV-LED 172, a lens 174, and a lens 176 having an optical axis 178. The UV-LED 172, the lens 174, and the lens 176 may be similar to the UV-LED 158, the lens 160, and the lens 164 except that the UV-LED 172, the lens 174, and the lens 176 may be positioned in the reactor head 170 such that the optical axis 178 may be parallel to a longitudinal direction of a reaction chamber (such as the longitudinal direction 106 of the reaction chamber 104, for example). As a result, the reactor head 170 may direct UV radiation into a reaction chamber skewed laterally similarly to the reactor head 130 as described above with reference to FIG. 2, for example.

Referring to FIG. 6, a reactor head according to another embodiment is shown generally at 180 and includes a UV-LED 182, a lens 184 having an optical axis 186, and a lens 188 having an optical axis 190. In the embodiment shown, the lens 184 is a half-ball lens and the lens 188 is a plano-convex lens, although alternative embodiments may differ. At least some UV radiation from the UV-LED 182 may be refracted by the lens 184, at least some UV radiation refracted by the lens 184 may be refracted by the lens 188, and at least some UV radiation refracted by the lens 188 may be directed into a reaction chamber, for example through the translucent or transparent wall 124 and into the reaction chamber 104 from the longitudinal end 108 or through the translucent or transparent wall 126 and into the reaction chamber 104 from the longitudinal end 110, or more generally into one or both longitudinal ends of a reaction chamber such as reaction chambers as described herein. Therefore, the UV-LED 182, the lens 184, and the lens 188 may collectively function as a UV source (or, more generally, as an electromagnetic radiation source) for a reaction chamber such as reaction chambers described herein, for example.

The UV radiation from the UV-LED 182 may be substantially axially symmetric about a principal radiation direction 192, and the optical axes 186 and 190 are parallel to and spaced apart from the principal radiation direction 192. In the embodiment shown, a separation distance between the optical axes 186 and 190 and the principal radiation direction 192 may be about 1% to about 37.5% of a diameter of the lens 184, although alternative embodiments may differ. As a result, as with the reactor head 156, UV radiation refracted by the lens 188 is not substantially axially symmetric about the principal radiation direction 192, but is rather skewed laterally relative to the principal radiation direction 192 and skewed laterally relative to the UV radiation refracted from the UV-LED 182. In other words, a fluence rate or local intensity of the UV radiation from the UV-LED 182 and refracted by the lenses 184 and 188 is greater on one transverse side of the principal radiation direction 192 (above the principal radiation direction 192 in the orientation of FIG. 6) than on an opposite transverse side of the principal radiation direction 192 (below the principal radiation direction 192 in the orientation of FIG. 6). Further, UV radiation refracted by the lens 188 may be refracted into a reaction chamber, and UV radiation refracted by the lens 188 and into a reaction chamber may be skewed laterally relative to a longitudinal direction of the reaction chamber, similarly to the reactor head 130 as described above with reference to FIG. 2, for example.

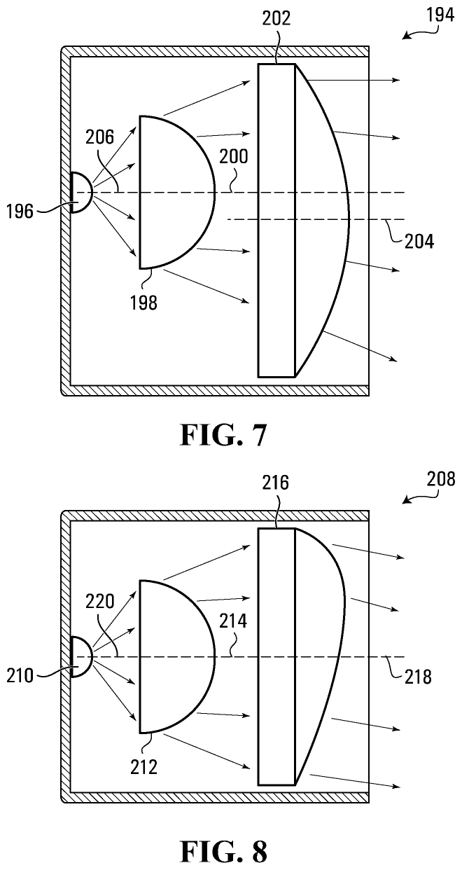

Referring to FIG. 7, a reactor head according to another embodiment is shown generally at 194 and includes a UV-LED 196, a lens 198 having an optical axis 200, and a lens 202 having an optical axis 204. In the embodiment shown, the lens 198 is a half-ball lens and the lens 202 is a plano-convex lens, although alternative embodiments may differ. At least some UV radiation from the UV-LED 196 may be refracted by the lens 198, at least some UV radiation refracted by the lens 198 may be refracted by the lens 202, and at least some UV radiation refracted by the lens 202 may be directed into a reaction chamber, for example through the translucent or transparent wall 124 and into the reaction chamber 104 from the longitudinal end 108 or through the translucent or transparent wall 126 and into the reaction chamber 104 from the longitudinal end 110, or more generally into one or both longitudinal ends of a reaction chamber such as reaction chambers as described herein. Therefore, the UV-LED 196, the lens 198, and the lens 202 may collectively function as a UV source (or, more generally, as an electromagnetic radiation source) for a reaction chamber such as reaction chambers described herein, for example.

The UV radiation from the UV-LED 196 may be substantially axially symmetric about a principal radiation direction 206, and the optical axis 200 is substantially collinear with the principal radiation direction 206, although alternative embodiments may differ. However, the optical axis 204 is parallel to and spaced apart from the principal radiation direction 206 and from the optical axis 200. In the embodiment shown, a separation distance between the optical axis 204 and the optical axis 200 may be about 1% to about 37.5% of a diameter of the lens 198, although alternative embodiments may differ. As a result, as with the reactor head 156, UV radiation refracted by the lens 202 is not substantially axially symmetric about the principal radiation direction 206, but is rather skewed laterally relative to the principal radiation direction 206 and skewed laterally relative to the UV radiation refracted from the UV-LED 206. In other words, a fluence rate or local intensity of the UV radiation from the UV-LED 196 and refracted by the lenses 198 and 202 is greater on one transverse side of the principal radiation direction 206 (above the principal radiation direction 206 in the orientation of FIG. 7) than on an opposite transverse side of the principal radiation direction 206 (below the principal radiation direction 206 in the orientation of FIG. 7). Further, UV radiation refracted by the lens 202 may be refracted into a reaction chamber, and UV radiation refracted by the lens 202 and into a reaction chamber may be skewed laterally relative to a longitudinal direction of the reaction chamber, similarly to the reactor head 130 as described above with reference to FIG. 2, for example.

Referring to FIG. 8, a reactor head according to another embodiment is shown generally at 208 and includes a UV-LED 210, a lens 212 having an optical axis 214, and a lens 216 having an optical axis 218. In the embodiment shown, the lens 212 is a half-ball lens and the lens 216 is a plano-convex lens, although alternative embodiments may differ. At least some UV radiation from the UV-LED 210 may be refracted by the lens 212, at least some UV radiation refracted by the lens 212 may be refracted by the lens 216, and at least some UV radiation refracted by the lens 216 may be directed into a reaction chamber, for example through the translucent or transparent wall 124 and into the reaction chamber 104 from the longitudinal end 108 or through the translucent or transparent wall 126 and into the reaction chamber 104 from the longitudinal end 110, or more generally into one or both longitudinal ends of a reaction chamber such as reaction chambers as described herein. Therefore, the UV-LED 210, the lens 212, and the lens 216 may collectively function as a UV source (or, more generally, as an electromagnetic radiation source) for a reaction chamber such as reaction chambers described herein, for example. The UV radiation from the UV-LED 210 may be substantially axially symmetric about a principal radiation direction 220, and the optical axes 214 and 218 are substantially collinear with the principal radiation direction 220, although alternative embodiments may differ. However, the lens 216 is axially asymmetric. As a result, as with the reactor head 156, UV radiation refracted by the lens 216 is not substantially axially symmetric about the principal radiation direction 220, but is rather skewed laterally relative to the principal radiation direction 220 and skewed laterally relative to the UV radiation refracted from the UV-LED 210. In other words, a fluence rate or local intensity of the UV radiation from the UV-LED 210 and refracted by the lenses 212 and 218 is greater on one transverse side of the principal radiation direction 220 (above the principal radiation direction 220 in the orientation of FIG. 8) than on an opposite transverse side of the principal radiation direction 220 (below the principal radiation direction 220 in the orientation of FIG. 8). Further, UV radiation refracted by the lens 216 may be refracted into a reaction chamber, and UV radiation refracted by the lens 216 and into a reaction chamber may be skewed laterally relative to a longitudinal direction of the reaction chamber, similarly to the reactor head 130 as described above with reference to FIG. 2, for example.

The reactor heads of FIGS. 3 to 8 are examples only, and alternative embodiments may differ. For example, each of the reactor heads of FIGS. 3 to 8 includes a UV-LED, but alternative embodiments may include more than one UV-LED, one or more other LEDs, one or more other emitters of UV radiation that may not necessarily be LEDs or UV-LEDs, or one or more emitters of electromagnetic radiation that may not necessarily be emitters of UV radiation. Further, each of the reactor heads of FIGS. 3 to 8 includes two lenses, but alternative embodiments may include fewer or more than two lenses. Further, in some embodiments, at least one lens may be incorporated into one or more electromagnetic radiation emitters, and at least one lens may be separate from one or more electromagnetic radiation emitters.

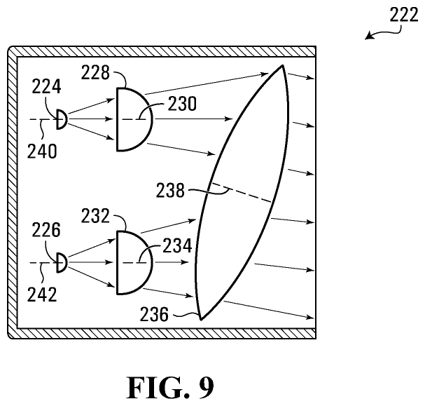

As another example, referring to FIG. 9, a reactor head according to another embodiment is shown generally at 222 and includes UV-LEDs 224 and 226, a lens 228 having an optical axis 230, a lens 232 having an optical axis 234, and a lens 236 having an optical axis 238. In the embodiment shown, the lenses 228 and 232 are half-ball lenses and the lens 236 is a biconvex or convex lens, although alternative embodiments may differ. At least some UV radiation from the UV-LED 224 may be refracted by the lens 228, at least some UV radiation refracted by the lens 228 may be refracted by the lens 236, and at least some UV radiation refracted by the lens 228 and by the lens 236 may be directed into a reaction chamber, for example through the translucent or transparent wall 124 and into the reaction chamber 104 from the longitudinal end 108 or through the translucent or transparent wall 126 and into the reaction chamber 104 from the longitudinal end 110, or more generally into one or both longitudinal ends of a reaction chamber such as reaction chambers as described herein.

Therefore, the UV-LEDs 224 and 226 and the lenses 228, 232, and 236 may collectively function as a UV source (or, more generally, as an electromagnetic radiation source) for a reaction chamber such as reaction chambers described herein, for example. Further, at least some UV radiation from the UV-LED 226 may be refracted by the lens 232, at least some UV radiation refracted by the lens 232 may be refracted by the lens 236, and at least some UV radiation refracted by the lens 232 and by the lens 236 may be directed into the same reaction chamber.

The UV radiation from the UV-LED 224 may be substantially axially symmetric about a principal radiation direction 240, and the optical axis 230 is substantially collinear with the principal radiation direction 240, although alternative embodiments may differ. Further, the

UV radiation from the UV-LED 226 may be substantially axially symmetric about a principal radiation direction 242, and the optical axis 234 is substantially collinear with the principal radiation direction 242, although again alternative embodiments may differ. However, the optical axis 238 is non-parallel and oblique to the principal radiation directions 240 and 242 and to the optical axes 230 and 234. In the embodiment shown, an oblique angle between the optical axis 238 and the principal radiation directions 240 and 242 (or between the optical axis 238 and a longitudinal direction of a reaction chamber, such as the longitudinal direction 106 of the reaction chamber 104, for example) may be between about 1 degree and about 45 degrees, although alternative embodiments may differ.

As a result, as with the reactor head 156, UV radiation refracted by the lens 238 is not substantially axially symmetric about the principal radiation direction 240 or 242, but is rather skewed laterally relative to the principal radiation directions 240 and 242 and skewed laterally relative to the UV radiation refracted from the UV-LEDs 224 and 226. In other words, a fluence rate or local intensity of the UV radiation from the UV-LEDs 224 and 226 and refracted by the lenses 228, 232, and 236 is greater on one transverse side of the principal radiation directions 240 and 242 (above the principal radiation directions 240 and 242 in the orientation of FIG. 9) than on an opposite transverse side of the principal radiation directions 240 and 242 (below the principal radiation directions 240 and 242 in the orientation of FIG. 9). Further, UV radiation refracted by the lens 236 may be refracted into a reaction chamber, and UV radiation refracted by the lens 236 and into a reaction chamber may be skewed laterally relative to a longitudinal direction of the reaction chamber, similarly to the reactor head 130 as described above with reference to FIG. 2, for example.

Again, the reactor head of FIG. 9 is an example only, and alternative embodiments may differ. For example, the reactor head of FIG. 9 includes two UV-LEDs, but alternative embodiments may include fewer or more UV-LEDs, one, two, or more than two other LEDs, one, two, or more than two other emitters of UV radiation that may not necessarily be UV-LEDs, or one, two, or more than two emitters of electromagnetic radiation that may not necessarily be emitters of UV radiation. Further, the reactor head of FIG. 9 includes three lenses, but alternative embodiments may include fewer or more than three lenses. Further, in some embodiments, at least one lens may be incorporated into one or more electromagnetic radiation emitters, and at least one lens may be separate from one or more electromagnetic radiation emitters. In general, lenses as described herein may be configured to refract electromagnetic radiation from different emitters of electromagnetic radiation such as those described herein, for example.

Further, similar to the embodiment of FIG. 4, the UV-LEDs 224 and 226 and the lenses 228, 232, and 236 may be positioned in the reactor head 222 such that the principal radiation directions 240 and 242 and the optical axes 230 and 234 may be parallel to a longitudinal direction of a reactor (such as the longitudinal direction 106 of the reaction chamber 104, for example), but alternative embodiments may differ. For example, similar to the embodiment of FIG. 5, the UV-LEDs 224 and 226 and the lenses 228, 232, and 236 may be positioned in the reactor head 222 such that the optical axis 238 may be parallel to such a longitudinal direction of a reactor, and still the reactor head 222 may direct UV radiation into a reaction chamber skewed laterally similarly to the reactor head 130 as described above, for example.

The reactor heads of FIGS. 3 to 9 are examples only, and in general, in different embodiments, electromagnetic radiation (such as UV radiation, for example) may be refracted by at least one lens having an optical axis non-parallel to a longitudinal direction of a reaction chamber (such as the longitudinal direction 106 of the reaction chamber 104, for example), by at least one lens having an optical axis non-parallel to a principal radiation direction of an emitter of electromagnetic radiation (such as the principal radiation direction 168, 192, 206, or 220, for example), by at least one lens having an optical axis parallel to and spaced apart from the principal radiation direction, by at least one axially asymmetric lens, by one or more other lenses, or by a combination of two or more thereof.

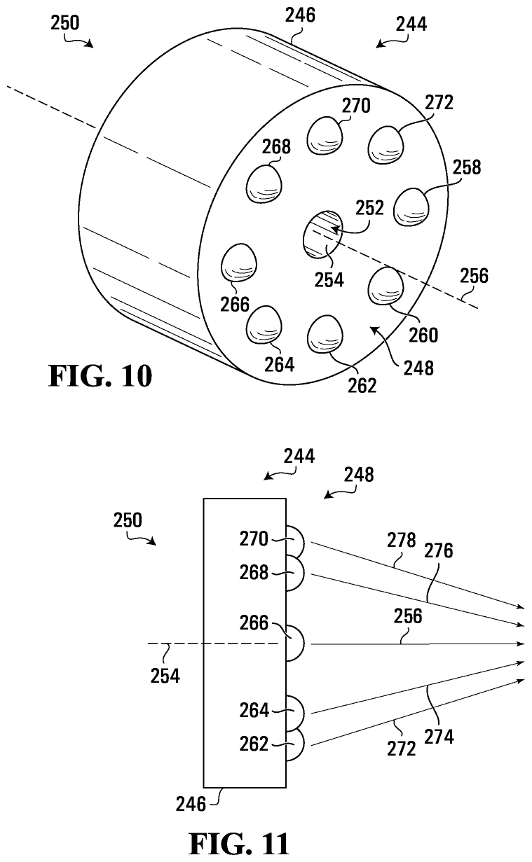

Reactor heads according to other embodiments may define one or more fluid conduits that may function as inlets or outlets to reaction chambers. Further, reactor heads according to other embodiments may include more than one electromagnetic radiation emitter. For example, referring to FIGS. 10 and 11, a reactor head according to another embodiment is shown generally at 244 and includes a body 246 having opposite sides 248 and 250. The body 246 defines a fluid conduit 252 extending between the opposite sides 248 and 250. The fluid conduit 252 extends along an axis 254 and may function as an inlet or as an outlet to a reaction chamber. Therefore, if the fluid conduit 252 functions as an inlet to a reaction chamber, then the fluid conduit 252 is configured to direct fluid into the reaction chamber in an inlet direction 256 that may be an extension of the axis 254 into the reaction chamber. Likewise, if the fluid conduit 252 functions as an outlet to a reaction chamber, then the fluid conduit 252 is configured to direct fluid out of the reaction chamber in an outlet direction that may be an extension of the axis 254. The reactor head 244 also includes electromagnetic radiation sources 258, 260, 262, 264, 266, 268, 270, and 272, although alternative embodiments may include more or fewer electromagnetic radiation sources. Each electromagnetic radiation source may include one or more electromagnetic radiation emitters and one or more lenses such as those described above, for example, although for simplicity, FIGS. 10 and 11 illustrate only outermost lenses of the electromagnetic radiation emitters. In the embodiment shown, the electromagnetic radiation sources 258, 260, 262, 264, 266, 268, 270, and 272 are on the side 248 and surround the fluid conduit 252, although alternative embodiments may differ.

The electromagnetic radiation sources 258, 260, 262, 264, 266, 268, 270, and 272 may be similar to electromagnetic radiation sources as described above and as illustrated in FIGS. 3 to 9, for example, and may therefore produce electromagnetic radiation (such as UV radiation, for example) skewed laterally as illustrated in FIGS. 3 to 9, for example. Further, in the embodiment shown, the electromagnetic radiation sources 258, 260, 262, 264, 266, 268, 270, and 272 may produce electromagnetic radiation skewed laterally towards the inlet direction 256. For example, as shown in FIG. 11, a principal radiation direction 272 of electromagnetic radiation from the electromagnetic radiation source 262 is skewed laterally towards the inlet direction 256, a principal radiation direction 274 of electromagnetic radiation from the electromagnetic radiation source 264 is skewed laterally towards the inlet direction 256, a principal radiation direction 276 of electromagnetic radiation from the electromagnetic radiation source 268 is skewed laterally towards the inlet direction 256, and a principal radiation direction 278 of electromagnetic radiation from the electromagnetic radiation source 270 is skewed laterally towards the inlet direction 256. The principal radiation directions 272, 274, 276, and 278 are shown in FIG. 11 for simplicity, but principal radiation directions of other electromagnetic radiation sources of the reactor head 244 may also be skewed laterally towards the inlet direction 256. In other words, the reactor head 244 includes a plurality of lenses (namely lenses of the electromagnetic radiation sources 258, 260, 262, 264, 266, 268, 270, and 272) that are spaced apart around, and that surround, an axis that extends along the inlet direction 256 and that are configured to cause refracted electromagnetic radiation to be skewed laterally towards an extension of the inlet direction 256, although alternative embodiments may differ.

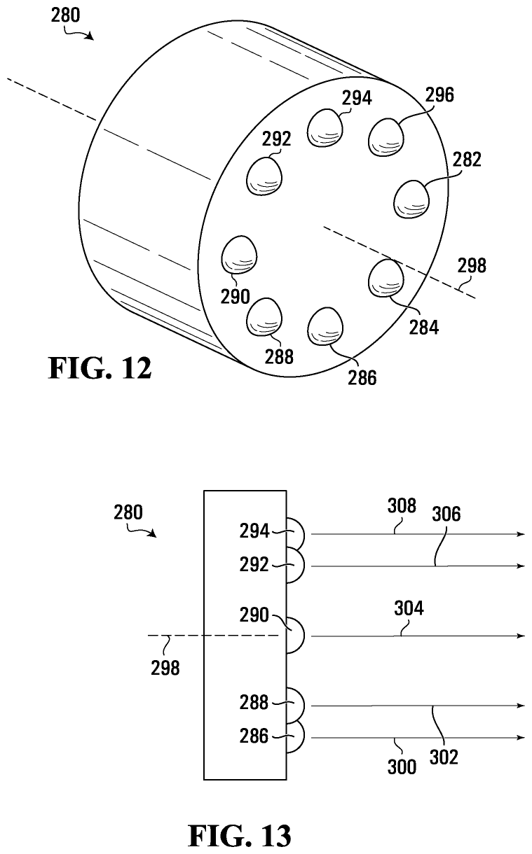

Referring to FIGS. 12 and 13, a reactor head according to another embodiment is shown generally at 280 and includes electromagnetic radiation sources 282, 284, 286, 288, 290, 292, 294, and 296, although alternative embodiments may include more or fewer electromagnetic radiation sources. Again, each electromagnetic radiation source may include one or more electromagnetic radiation emitters and one or more lenses such as those described above, for example, although for simplicity, FIGS. 12 and 13 illustrate only outermost lenses of the electromagnetic radiation emitters. Also, in the embodiment shown, the electromagnetic radiation sources 282, 284, 286, 288, 290, 292, 294, and 296 surround a central axis 298 of the reactor head 280, although alternative embodiments may differ.

The electromagnetic radiation sources 282, 284, 286, 288, 290, 292, 294, and 296 may be similar to the UV-LED 132, the lens 134, and the lens 136 shown in FIG. 2. Therefore, the electromagnetic radiation sources 282, 284, 286, 288, 290, 292, 294, and 296 may produce electromagnetic radiation (such as UV radiation, for example) that is substantially collimated or that diverges, and principal radiation directions of electromagnetic radiation produced by the electromagnetic radiation sources 282, 284, 286, 288, 290, 292, 294, and 296 may be substantially parallel to the central axis 298 of the reactor head 280. For example, as shown in FIG. 13, a principal radiation direction 300 of electromagnetic radiation from the electromagnetic radiation source 286 is substantially parallel to the central axis 298, a principal radiation direction 302 of electromagnetic radiation from the electromagnetic radiation source 288 is substantially parallel to the central axis 298, a principal radiation direction 304 of electromagnetic radiation from the electromagnetic radiation source 290 is substantially parallel to the central axis 298, a principal radiation direction 306 of electromagnetic radiation from the electromagnetic radiation source 292 is substantially parallel to the central axis 298, and a principal radiation direction 308 of electromagnetic radiation from the electromagnetic radiation source 294 is substantially parallel to the central axis 298. The principal radiation directions 300, 302, 304, 306, and 308 are shown in FIG. 13 for simplicity, but principal radiation directions of other electromagnetic radiation sources of the reactor head 280 may also be substantially parallel to the central axis 298.

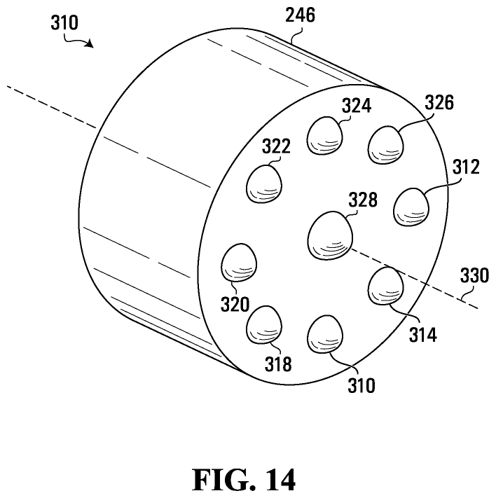

Referring to FIG. 14, a reactor head according to another embodiment is shown generally at 310 and includes electromagnetic radiation sources 312, 314, 316, 318, 320, 322, 324, 326, and 328, although alternative embodiments may include more or fewer electromagnetic radiation sources. The electromagnetic radiation sources 312, 314, 316, 318, 320, 322, 324, and 326 may be similar to the electromagnetic radiation sources 282, 284, 286, 288, 290, 292, 294, and 296 as described above and surround a central axis 330 of the reactor head 310, although alternative embodiments may differ. Further, like the electromagnetic radiation sources 282, 284, 286, 288, 290, 292, 294, and 296, the electromagnetic radiation sources 312, 314, 316, 318, 320, 322, 324, and 326 may produce electromagnetic radiation (such as UV radiation, for example) that is substantially collimated or that diverges, and principal radiation directions of electromagnetic radiation produced by the electromagnetic radiation sources 312, 314, 316, 318, 320, 322, 324, and 326 may be substantially parallel to the central axis 330 of the reactor head 310.

The electromagnetic radiation source 328 may be positioned along the central axis 330 so that the electromagnetic radiation sources 312, 314, 316, 318, 320, 322, 324, and 326 also surround the electromagnetic radiation source 328. Like the electromagnetic radiation sources 312, 314, 316, 318, 320, 322, 324, and 326, the electromagnetic radiation source 328 may also produce electromagnetic radiation (such as UV radiation, for example) that is substantially collimated or that diverges, and a principal radiation direction of electromagnetic radiation produced by the electromagnetic radiation source 328 may also be substantially parallel to the central axis 330 of the reactor head 310. In some embodiments, the electromagnetic radiation source 328 may be larger and/or may produce electromagnetic radiation at more power or intensity than the electromagnetic radiation sources 312, 314, 316, 318, 320, 322, 324, and 326 individually.

In general, reactor heads such as those described above may direct electromagnetic radiation (such as UV radiation, for example) into different reaction chambers of different reactor apparatuses. In some embodiments, such reaction chambers may have longitudinal ends, and such reactor heads may be positioned to direct electromagnetic radiation into such reaction chambers from one or both of such longitudinal ends.

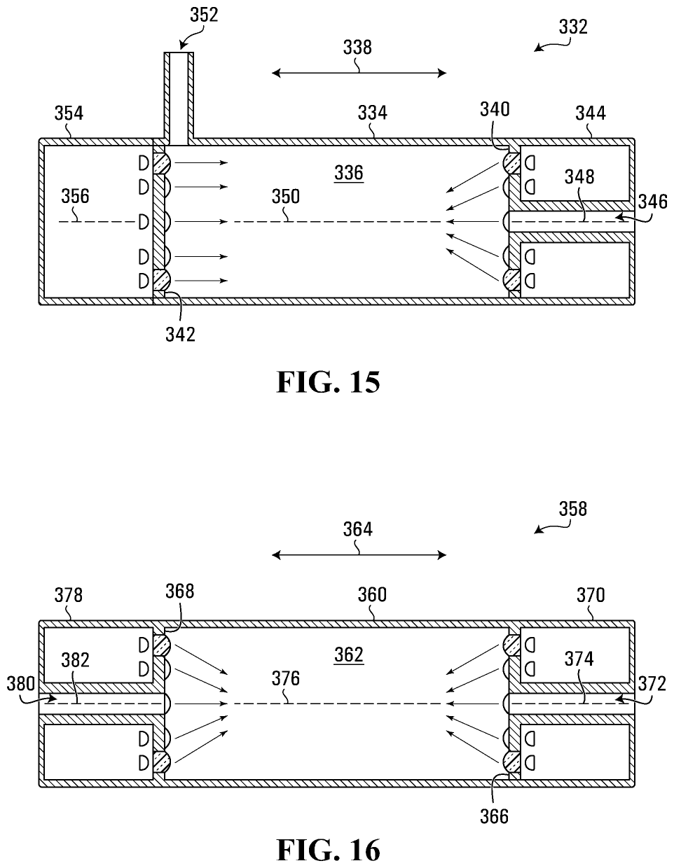

For example, referring to FIG. 15, a reactor apparatus according to another embodiment is shown generally at 332 and includes a reactor body 134 that defines a reaction chamber 336 that extends in a longitudinal direction 338 between longitudinal ends 340 and 342 of the reaction chamber 336.

The reactor apparatus 332 also includes a reactor head 344 proximate the longitudinal end 340 and positioned to direct electromagnetic radiation into the reaction chamber 336 from the longitudinal end 340. The reactor head 344 may be similar to the reactor head 244.

Therefore, the reactor head 344 defines an inlet 346 to the reaction chamber 336 proximate the longitudinal end 340, the inlet 346 extends along an inlet axis 348, and the inlet 346 is configured to direct fluid into the reaction chamber 336 in an inlet direction that may be an extension of the inlet axis 348 into the reaction chamber 336. In the embodiment shown, the inlet axis 348 and the inlet direction are substantially collinear with or parallel to a central longitudinal axis 350 of the reaction chamber 336 extending in the longitudinal direction 338, but alternative embodiments may differ.

Fluid in the reaction chamber 336 may flow faster in regions of the reaction chamber 336 that are downstream from the inlet 346 than in other regions of the reaction chamber 336. Also, because the reactor head 344 may be similar to the reactor head 244, principal radiation directions of electromagnetic radiation sources of the reactor head 344 may also be skewed laterally towards the inlet direction and thus towards the central longitudinal axis 350 of the reaction chamber 336, as shown in FIG. 15, but again alternative embodiments may differ.

Because fluid in the reaction chamber 336 may flow faster in regions of the reaction chamber 336 that are downstream from the inlet 346 than in other regions of the reaction chamber 336, and because principal radiation directions of electromagnetic radiation sources of the reactor head 344 may be skewed laterally towards the inlet direction as shown in FIG. 15, a UV fluence rate (density of UV intensity) or local UV intensity in the reaction chamber 336 may, in general, be higher in regions where fluid flow velocity in the reaction chamber 336 may also be higher, and total UV exposure to fluid flowing through the reaction chamber 336 may be more consistent than in other reactor apparatuses without such skewed UV radiation.

The reactor body 334 also defines an outlet 352 of the reaction chamber 336 proximate the longitudinal end 342. The reaction chamber 336 therefore extends in the longitudinal direction 338 at least between the inlet 346 and the outlet 352.

The reactor apparatus 332 also includes a reactor head 354 proximate the longitudinal end 342 and positioned to direct electromagnetic radiation into the reaction chamber 336 from the longitudinal end 342. The reactor head 354 may be similar to the reactor head 280 or the reactor head 310. Therefore, electromagnetic radiation from electromagnetic radiation sources of the reactor head 354 may be substantially collimated or may be divergent, and principal radiation directions of electromagnetic radiation sources of the reactor head 354 may be substantially parallel to a central axis 356 of the reactor head 354, as shown in FIG. 15, but again alternative embodiments may differ. In the embodiment shown, the central axis 356 of the reactor head 354 is substantially collinear with or parallel to the central longitudinal axis 350 of the reaction chamber 336, so the principal radiation directions of electromagnetic radiation sources of the reactor head 354 may be substantially parallel to the central longitudinal axis 350 of the reaction chamber 336, but again alternative embodiments may differ.

Referring to FIG. 16, a reactor apparatus according to another embodiment is shown generally at 358 and includes a reactor body 360 that defines a reaction chamber 362 that extends in a longitudinal direction 364 between longitudinal ends 366 and 368 of the reaction chamber 362.

The reactor apparatus 358 also includes a reactor head 370 proximate the longitudinal end 366 and positioned to direct electromagnetic radiation into the reaction chamber 362 from the longitudinal end 366. The reactor head 370 may be similar to the reactor head 244 and defines an inlet 372 to the reaction chamber 362 proximate the longitudinal end 366. Therefore, the inlet 372 extends along an inlet axis 374, and the inlet 372 is configured to direct fluid into the reaction chamber 362 in an inlet direction that may be an extension of the inlet axis 374 into the reaction chamber 362. In the embodiment shown, the inlet axis 374 and the inlet direction are substantially collinear with or parallel to a central longitudinal axis 376 of the reaction chamber 362 extending in the longitudinal direction 364, but alternative embodiments may differ. Because the reactor head 370 may be similar to the reactor head 244, principal radiation directions of electromagnetic radiation sources of the reactor head 370 may also be skewed laterally towards the inlet direction and thus towards the central longitudinal axis 376 of the reaction chamber 362, as shown in FIG. 16, but again alternative embodiments may differ.

The reactor apparatus 358 also includes a reactor head 378 proximate the longitudinal end 368 and positioned to direct electromagnetic radiation into the reaction chamber 362 from the longitudinal end 368. The reactor head 378 may be similar to the reactor head 244 and defines an outlet 380 to the reaction chamber 362 proximate the longitudinal end 366. Therefore, the reaction chamber 362 extends in the longitudinal direction 364 at least between the inlet 372 and the outlet 380. Further, the outlet 380 extends along an outlet axis 382. In the embodiment shown, the outlet axis 382 is substantially collinear with or parallel to the central longitudinal axis 376 of the reaction chamber 362, but alternative embodiments may differ. Because the reactor head 378 may be similar to the reactor head 244, principal radiation directions of electromagnetic radiation sources of the reactor head 378 may also be skewed laterally towards the central longitudinal axis 376 of the reaction chamber 362, as shown in FIG. 16, but again alternative embodiments may differ.

Fluid in the reaction chamber 362 may flow faster in regions of the reaction chamber 362 that are downstream from the inlet 372 and that are upstream from the outlet 380 than in other regions of the reaction chamber 362. Because principal radiation directions of electromagnetic radiation sources of the reactor heads 370 and 378 may be skewed laterally towards the central longitudinal axis 376 of the reaction chamber 362, as shown in FIG. 16, UV radiation fluence rate or local intensity in the reaction chamber 362 may, in general, be higher in regions where fluid flow velocity in the reaction chamber 362 may also be higher, and total UV exposure to fluid flowing through the reaction chamber 362 may be more consistent than in other reactor apparatuses without such skewed UV radiation.

The reactor apparatuses and reactor heads described above are examples only, and alternative embodiments may differ. For example, reactor heads according to alternative embodiments may include different combinations of one or more electromagnetic radiation emitters and one or more lenses that may skew electromagnetic radiation from the one or more electromagnetic radiation emitters laterally similarly to the embodiments described above, or in different ways.

Further, reactor apparatuses according to alternative embodiments may have one or more inlets, one or more outlets, one or more reaction chambers, and one or more reactor heads that may be similar to the embodiments described above, or that may vary in different ways. For example, reactor apparatuses according to alternative embodiments may define one or more than one reaction chamber, and may include one, two, or more than two reactor heads such as those described herein positioned to direct electromagnetic radiation into each such reaction chamber.

In general, embodiments such as those described herein may involve laterally skewed electromagnetic radiation in a reaction chamber such that UV radiation fluence rate or local intensity in the reaction chamber may, in general, be higher in regions where fluid flow velocity in the reaction chamber may also be higher, and total UV exposure to fluid flowing through the reaction chamber may be more consistent than in other reactor apparatuses without such skewed UV radiation. Such relatively more consistent total UV exposure may enhance treatment of fluid that flows in the reaction chamber and may, for example, deactivate pathogens in the fluid more effectively than in other reactor apparatuses without such skewed UV radiation.

Although specific embodiments have been described and illustrated, such embodiments should be considered illustrative only and not as limiting the invention as construed according to the accompanying claims.

1. A method of distributing electromagnetic radiation in a reaction chamber extending in a longitudinal direction at least between an inlet of the reaction chamber and an outlet of the reaction chamber, the method comprising:
causing at least some electromagnetic radiation from at least one electromagnetic radiation emitter to be refracted by at least one lens into the reaction chamber as refracted electromagnetic radiation skewed laterally relative to the longitudinal direction.

2. A method of distributing electromagnetic radiation in a reaction chamber, the method comprising:
causing at least some electromagnetic radiation from at least one electromagnetic radiation emitter to be refracted by at least one lens into the reaction chamber as refracted electromagnetic radiation skewed laterally relative to the at least some electromagnetic radiation from the at least one electromagnetic radiation emitter.

3. The method of claim 2 wherein the reaction chamber extends in a longitudinal direction at least between an inlet of the reaction chamber and an outlet of the reaction chamber.

4. The method of claim 3 wherein causing the at least some of the refracted electromagnetic radiation to be refracted into the reaction chamber comprises causing the refracted electromagnetic radiation in the reaction chamber to be skewed laterally relative to the longitudinal direction.

5. The method of claim 1, 3, or 4 wherein the longitudinal direction is parallel to a central longitudinal axis of the reaction chamber.

6. The method of any one of claim 1, 3, 4, or 5 wherein the inlet is configured to direct fluid into the reaction chamber in an inlet direction non-parallel to the longitudinal direction.

7. The method of claim 6 wherein the inlet direction is substantially perpendicular to the longitudinal direction.

8. The method of claim 6 or 7 wherein causing the at least some of the refracted electromagnetic radiation to be refracted into the reaction chamber comprises causing fluence rate of the refracted electromagnetic radiation in the reaction chamber and along the inlet direction from the inlet to be higher with increased distance from the inlet.

9. The method of claim 6, 7, or 8 wherein causing the at least some of the refracted electromagnetic radiation to be refracted into the reaction chamber comprises causing a fluence rate of the refracted electromagnetic radiation in a first transverse side of the reaction chamber proximate the inlet to be less than a fluence rate of the refracted electromagnetic radiation in a second transverse side of the reaction chamber opposite the first transverse side of the reaction chamber and opposite the inlet.

10. The method of claim 1, 3, 4, or 5 wherein the inlet is configured to direct fluid into the reaction chamber in an inlet direction substantially parallel to the longitudinal direction.

11. The method of claim 10, when dependent directly or indirectly on claim 1 or 4, wherein causing the refracted electromagnetic radiation in the reaction chamber to be skewed laterally relative to the longitudinal direction comprises causing the refracted electromagnetic radiation in the reaction chamber to be skewed laterally relative to the longitudinal direction and towards an extension in the reaction chamber of the inlet direction from the inlet.

12. The method of claim 11 wherein causing the at least some electromagnetic radiation from the at least one electromagnetic radiation emitter to be refracted by the at least one lens into the reaction chamber comprises causing the at least some electromagnetic radiation from the at least one electromagnetic radiation emitter to be:
refracted into the reaction chamber by a plurality of lenses spaced apart around an inlet axis extending along the inlet direction; and
skewed laterally relative to the longitudinal direction and towards the extension in the reaction chamber of the inlet direction from the inlet.

13. The method of claim 12 wherein the plurality of lenses surround the inlet axis.

14. The method of any one of claims 1 to 13 wherein the electromagnetic radiation comprises ultraviolet (“UV”) radiation.

15. The method of claim 14 wherein the at least one electromagnetic radiation emitter comprises at least one UV light-emitting diode (“UV-LED”).

16. The method of any one of claims 1 to 13 wherein the at least one electromagnetic radiation emitter comprises at least one light-emitting diode (“LED”).

17. The method of any one of claims 1 to 16 wherein the at least some electromagnetic radiation from the at least one electromagnetic radiation emitter has a principal radiation direction.

18. The method of claim 17 wherein the at least some electromagnetic radiation from the at least one electromagnetic radiation emitter is substantially axially symmetric about the principal radiation direction.

19. The method of claim 17 or 18 wherein the refracted electromagnetic radiation is distributed axially asymmetrically relative to the principal radiation direction of the at least some electromagnetic radiation from the at least one electromagnetic radiation emitter.

20. The method of claim 17, 18, or 19 wherein a fluence rate of the refracted electromagnetic radiation on a first transverse side of the principal radiation direction is greater than a fluence rate of the refracted electromagnetic radiation on a second transverse side of the principal radiation direction opposite the first transverse side of the principal radiation direction.

21. The method of claim 17, 18, 19, or 20 wherein the at least one lens comprises at least one lens having an optical axis non-parallel to the principal radiation direction.

22. The method of claim 17, 18, 19, or 20 wherein the at least one lens comprises at least one lens having an optical axis parallel to and spaced apart from the principal radiation direction.

23. The method of any one of claims 1 to 20 wherein the at least one lens comprises at least one axially asymmetric lens.

24. A reactor apparatus comprising:
a body defining an inlet, an outlet, and a reaction chamber extending in a longitudinal direction at least between the inlet and the outlet;
at least one electromagnetic radiation emitter; and
at least one lens configured to refract at least some electromagnetic radiation from the at least one electromagnetic radiation emitter into the reaction chamber as refracted electromagnetic radiation skewed laterally relative to the longitudinal direction.

25. A reactor apparatus comprising:
a body defining a reaction chamber;
at least one electromagnetic radiation emitter; and
at least one lens configured to refract at least some electromagnetic radiation from the at least one electromagnetic radiation emitter into the reaction chamber as refracted electromagnetic radiation skewed laterally relative to the at least some electromagnetic radiation from the at least one electromagnetic radiation emitter and into the reaction chamber.

26. The apparatus of claim 25 wherein:
the body further defines an inlet of the reaction chamber and an outlet of the reaction chamber; and
the reaction chamber extends in a longitudinal direction at least between the inlet and the outlet.

27. The apparatus of claim 26 wherein the at least one lens is configured to cause the refracted electromagnetic radiation in the reaction chamber to be skewed laterally relative to the longitudinal direction.

28. The apparatus of claim 24, 26, or 27 wherein the longitudinal direction is parallel to a central longitudinal axis of the reaction chamber.

29. The apparatus of claim 24, 26, 27, or 28 wherein the inlet is configured to direct fluid into the reaction chamber in an inlet direction non-parallel to the longitudinal direction.

30. The apparatus of claim 29 wherein the inlet direction is substantially perpendicular to the longitudinal direction.

31. The apparatus of claim 29 or 30 wherein the at least one lens is configured to cause fluence rate of the refracted electromagnetic radiation in the reaction chamber and along the inlet direction from the inlet to be higher with increased distance from the inlet.

32. The apparatus of claim 29, 30, or 31 wherein the at least one lens is configured to cause a fluence rate of the refracted electromagnetic radiation in a first transverse side of the reaction chamber proximate the inlet to be less than a fluence rate of the refracted electromagnetic radiation in a second transverse side of the reaction chamber opposite the first transverse side of the reaction chamber and opposite the inlet.

33. The apparatus of claim 24, 26, 27, or 28 wherein the inlet is configured to direct fluid into the reaction chamber in an inlet direction substantially parallel to the longitudinal direction.

34. The apparatus of claim 33, when dependent directly or indirectly on claim 24 or 27, wherein the at least one lens is configured to cause the refracted electromagnetic radiation in the reaction chamber to be skewed laterally relative to the longitudinal direction and towards an extension in the reaction chamber of the inlet direction from the inlet.

35. The apparatus of claim 34 wherein the at least one lens comprises a plurality of lenses spaced apart around an inlet axis extending along the inlet direction.

36. The apparatus of claim 35 wherein the plurality of lenses surround the inlet axis.

37. The apparatus of any one of claims 24 to 36 wherein the at least one electromagnetic radiation emitter comprises at least one emitter of UV radiation.

38. The apparatus of claim 37 wherein the at least one emitter of UV radiation comprises at least one UV-LED.

39. The apparatus of any one of claims 24 to 36 wherein the at least one electromagnetic radiation emitter comprises at least one LED.

40. The apparatus of any one of claims 24 to 39 wherein the at least one electromagnetic radiation emitter is configured to cause the at least some electromagnetic radiation from the at least one electromagnetic radiation emitter to have a principal radiation direction.

41. The apparatus of claim 40 wherein the at least one electromagnetic radiation emitter is configured to cause the at least some electromagnetic radiation from the at least one electromagnetic radiation emitter to be substantially axially symmetric about the principal radiation direction.

42. The apparatus of claim 40 or 41 wherein the at least one lens is configured to cause the refracted electromagnetic radiation to be distributed axially asymmetrically relative to the principal radiation direction of the at least some electromagnetic radiation from the at least one electromagnetic radiation emitter.

43. The apparatus of claim 40, 41, or 42 wherein the at least one lens is configured to cause a fluence rate of the refracted electromagnetic radiation on a first transverse side of the principal radiation direction to be greater than a fluence rate of the refracted electromagnetic radiation on a second transverse side of the principal radiation direction opposite the first transverse side of the principal radiation direction.

44. The apparatus of claim 40, 41, 42, or 43 wherein the at least one lens has an optical axis non-parallel to the principal radiation direction.

45. The apparatus of claim 40, 41, 42, or 43 wherein the at least one lens has an optical axis parallel to and spaced apart from the principal radiation direction.

46. The apparatus of any one of claims 24 to 43 wherein the at least one lens comprises an axially asymmetric lens.
